(19) United States
(12) Patent Application Publication
(10) Pub. No.: US 2025/0354194 A1
(43) Pub. Date: Nov. 20, 2025

(54) POLYDOPAMINE COATINGS FOR MICROORGANISM CAPTURE

(71) Applicant: Brigham Young University, Provo, UT (US)

(72) Inventors: Bowen J. Houser, Castle Rock, CO (US); William G. Pitt, Castle Rock, CO (US)

(21) Appl. No.: 19/208,283

(22) Filed: May 14, 2025

Related U.S. Application Data
(60) Provisional application No. 63/647,430, filed on May 14, 2024.

(51) Int. Cl. C12Q 1/24; B01J 20/26; B01J 20/28; B01L 3/00

(52) U.S. Cl. CPC C12Q 1/24; B01J 20/262; B01J 20/28007; B01J 20/28009; B01J 20/28016; B01L 3/502761; B01L 2200/0647; B01L 2300/16

(57) ABSTRACT

According to some embodiments, a method for extracting microorganisms from an extraction target is provided. In some cases, the method includes forming a polymerized pDA coating on a substrate. In some cases, the pDA coating includes at least one of: (a) dopamine; and (b) a dopamine substitute. In some embodiments, the method includes exposing the pDA coating on the substrate to a fluid containing the microorganisms such that at least a portion of the microorganisms bind to the pDA coating. In some cases, the method includes removing the pDA-coated substrate together with the portion of the microorganisms that bound to the pDA coating from the fluid. Other implementations are described.

### RELATED APPLICATIONS AND PRIORITY CLAIM

This application claims priority to U.S. Provisional Patent Application No. 63/647,430, entitled “ADHESION OF BACTERIA TO PARTICLES”, filed May 14, 2024 (Attorney Docket No. 2024-022)—the contents of which are incorporated herein by reference in their entirety.

### FIELD

The present disclosure relates to microorganism capture and analysis, and more particularly to compositions for binding microorganisms, such as bacteria, for capture and rapid analysis.

### BACKGROUND

Many fields of endeavor benefit from microorganism analytics. For example, the safety, efficacy, reliability, and usefulness of medical diagnostics, food processing, and many other processes may be greatly increased when microorganisms can be accurately detected and characterized. Unfortunately, some microorganism analytics present a whole host of unique challenges. Extracting bacteria, for instance, can be challenging, time consuming, and expensive. Furthermore, once bacteria have been extracted from a media, isolating specific bacteria or species of bacteria can further complicate the process. Moreover, identifying or characterizing microorganisms—even once such microorganisms have been extracted and isolated—can lead to further efforts and costs.

In some cases, microorganisms are found in a medium (e.g., blood, water, or another medium) in dilute concentrations. Accordingly, traditional methods often require culturing of such bacteria for many hours to produce sufficient numbers or concentrations for accurate analysis. Such culturing can lead to unrepresentative bacterial ratios, or even the loss of some species altogether. Moreover, not only can culturing be labor-intensive and expensive, but the time required for such culturing can lead to life-threatening delays in medical treatment or other serious issues.

Thus, while techniques currently exist that are used to extract and characterize microorganisms, challenges still exist, including those listed above. Accordingly, it would be an improvement in the art to augment or even replace current techniques with other techniques.

### SUMMARY

In the areas of medical diagnostics, food processing, and other areas, there is keen interest in capturing and concentrating dilute suspensions of bacteria to provide a small but concentrated sample of bacteria for species identification, antibiotic susceptibility testing, and a variety of other purposes.

According to some implementations, systems and methods for extracting or capturing microorganisms from an extraction target are provided. In some cases, a polymerized polydopamine (or polydopamine substitute) coating (pDA coating) is formed on a substrate.

In some implementations, the pDA coating on the substrate is exposed to a fluid (e.g., liquid or gas) containing the microorganisms, such that at least a portion of the microorganisms bind to the pDA coating. In some cases, the pDA-coated substrate is optionally removed from the fluid (or vice versa), together with the portion of the microorganisms that are bound to the pDA coating. In some cases, the substrate comprises a countertop, wall, handle, knob, railing, wipe, mesh, filter, or any other suitable material or surface that may be exposed to microorganisms.

In some implementations, after the polymerized pDA coating is formed on the substrate (with the polymerized pDA coating including at least one of: dopamine and a dopamine substitute), the polymerized pDA coating on the substrate is exposed to microorganisms such that at least a portion of the microorganisms binds to the polymerized pDA coating. In some such implementations, the polymerized pDA coating and the substrate are collected (e.g., for testing or any other suitable purpose), together with the portion of the microorganisms that is bound to the polymerized pDA coating.

Additional implementations are also described.

### DESCRIPTION OF THE FIGURES

The objects and features of the present disclosure will become more fully apparent from the following description and appended claims, taken in conjunction with the accompanying figures. Understanding that these figures depict only some embodiments of the disclosed systems and methods (and are, therefore, not to be considered limiting in scope), such systems and methods will be described and explained with additional specificity and detail through the use of the accompanying figures in which:

FIG. 1A shows a scanning electron microscopy (SEM) image of magnetic nanoparticles (MNPs)—in particular, magnetic iron oxide particles—in accordance with some embodiments of the described systems and methods;

FIG. 1B shows an SEM image of polydopamine-(pDA)-coated MNPs (pDA-MNPs), in accordance with some embodiments;

FIG. 1C shows an SEM image of pDA-MNPs sputtered with metal (in particular, gold (Au) and palladium (Pd)), in accordance with some embodiments;

FIG. 1D shows a transmission electron microscopy (TEM) image of non-coated MNPs, in accordance with some embodiments;

FIG. 1E shows a TEM image of pDA-MNPs, in accordance with some embodiments;

FIG. 1F shows a closeup image of pDA-MNPs, with arrows indicating a pDA coating;

FIG. 1G shows another SEM image of MNPs, in accordance with some embodiments;

FIG. 1H shows another SEM image of pDA-MNPs, in line with some embodiments;

FIG. 2 shows a graph of the capture efficiency (CE) of MNPs created in accordance with some embodiments toward several bacterial strains, where the underlined circles mark the average capture by the pDA-coated MNPs (according to such embodiments), while the non-underlined circles mark the average capture by naked MNPs, and where the error bars represent the standard deviation of replicate experiments (n≥3);

FIG. 3 shows a graph of the CE of S. aureus as a function of an amount (in mg) of pDA-MNP beads per mL of bacterial suspension (in accordance with some embodiments), which suspension had 1.2×10⁸ CFU S. aureus in PBS;

FIG. 4 shows a graph of CE for 8 strains of bacteria on bare MNCs (▪) and pDA-coated MNCs (●), in accordance with some embodiments, where error bars represent standard deviations (n≥4), and overbars compare data for pDA-coated MNCs only—*: p=0.003. **: p=0.006;

FIG. 5 shows a graph of CE of S. epidermidis (▪), S. aureus (●), and S. mutans (▴), on pDA-MNPs after 10 minutes of binding time, as a function of the ratios of the numbers of MNPs to the numbers of bacterial CFUs, in accordance with some embodiments;

FIGS. 6A and 6B show SEM images of pDA-MNPs bound to S. aureus, where FIG. 6A shows a low-magnification magnification image (scale bar is 3 μm) showing several bacteria surrounded by clusters of MNPs, and FIG. 6B shows a magnified view (scale bar is 500 nm) of a bacterium as bound to the pDA-MNPs, in accordance with some embodiments; and

FIGS. 7A and 7B show graphs representing kinetics of bacterial capture by pDA-MNPs, in accordance with some embodiments (e.g., as described in Example 6), where FIG. 7A shows, in accordance with some embodiments, Kinetic curves of S. aureus at three different MNP final concentrations in the microfuge tube: 0.10 mg/mL (●), 0.26 mg/mL (♦) and 0.77 mg/mL (▪), producing ratios of MNP clusters to bacteria of 58,300 MNP/CFU, 146,000 MNP/CFU, and 439,000 MNP/CFU respectively, and where FIG. 7B shows, in accordance with some embodiments, Kinetic curves of S. epidermidis (▪) S. aureus (●), and S. mutans (▴), at 0.10 mg/mL of pDA-MNPs (58,300 MNP/CFU).

### DETAILED DESCRIPTION

A description of embodiments will now be given with reference to the figures. It is expected that the present systems and methods may take many other forms and shapes, hence the following disclosure is intended to be illustrative and not limiting, and the scope of the disclosure should be determined by reference to the appended claims.

As described above, some traditional methods for capturing microorganisms 24 (e.g., bacteria, viruses, fungi, archaea, protists, or any other microorganisms) can have many drawbacks, such as being expensive, difficult, labor-intensive, and time consuming (in some cases, requiring lengthy durations for culturing sparse microorganism extraction or carrying out other traditional processes). The systems and methods described herein address these and other drawbacks of many traditional microorganism analysis.

In many cases, fast and accurate bacterial analysis (or analysis of other microorganisms 24) is highly desirable. As examples, clinical diagnostics, food safety testing, environmental monitoring, potable water diagnostics (or purification), and many other fields frequently encounter situations in which microorganism analysis or sequestration is important. In healthcare, rapid identification of microorganism pathogens can guide immediate and appropriate treatment, improving patient outcomes and reducing the spread of infection. In food production and distribution, early detection of microorganism contamination can help prevent outbreaks and ensure consumer safety. Similarly, in environmental settings, prompt microorganism analysis can detect waterborne pathogens or biohazards, enabling swift corrective action to protect public health and ecosystems.

In line with the foregoing, some embodiments of the instant systems and methods include one or more coatings configured to bind one or more types of microorganisms 24 to aid in the capture (and in some cases, the further processing) of such microorganisms.

In accordance with some embodiments, the described systems and methods include one or more polydopamine (pDA) coatings 18. Although many embodiments of the pDA coating include dopamine (e.g., in a polymerized form), some embodiments of the pDA coating (as referenced herein) include one or more alternatives to, or derivatives of, dopamine (dopamine substitutes), such as one or more catecholamines (e.g., low-molecular-weight catecholamines), catechols, dopamine hydrochloride, N-acetyldopamine (NADA), levodopa (L-DOPA), dopamine methacrylamide (DMA), dopamine acrylamide, dopamine methacrylate, dopamine acrylate, dopamine-conjugated polyethylene glycol (PEG-dopamine), catechol-functionalized polyethylene glycol (catechol-PEG), dopamine sulfonate, 6-hydroxydopamine (6-OHDA), dopamine thiol derivatives (e.g., dopamine-thiol, dopamine-cysteine conjugates, or any other suitable dopamine thiol derivatives), DOPA, 3,4-dihyroxyphenylalanine, epinephrine, norepinephrine, 6-nitro-dopamine, 2-bromo-N-[2-(3,4-dihydroxyphenyl)ethyl]-2-methyl propenamide, hydrocaffeic acid, caffeic acid, ferulic acid, gallic acid, pyrocatechol, tyramine, L-tyrosine, and any other suitable dopamine substitute or combination of dopamine or dopamine substitutes. For the purposes of this disclosure, a coating that contains a dopamine substitute will (unless otherwise expressly stated, e.g., in the claims or elsewhere) be considered a pDA coating (whether or not it actually includes dopamine itself), although references to a pDA coating are sometimes also intended to refer to a coating that uses primarily (or solely) polymerized dopamine as the structural polymer. Accordingly (and unless expressly stated otherwise), the pDA coating can include any coating that includes a polymerized form of dopamine or a dopamine substitute.

In some embodiments, the pDA coating 18 includes at least: 20%, 30%, 50%, 80%, 90%, 95%, or 99% dopamine. In some cases, the pDA coating includes at least: 20%, 30%, 50%, 80%, 90%, 95%, or 99% dopamine substitute. In some embodiments, the pDA coating includes a combination of dopamine and a dopamine substitute. Indeed, some embodiments include at least 25% dopamine and at least 25% dopamine substitute. As discussed in more detail below, the pDA coating may be useful in binding certain types of microorganisms. In this regard, using different constituents (e.g., different combinations of dopamine and dopamine substitutes) in the pDA coating may cause the pDA to be more or less effective at binding certain types of microorganisms. Thus, various combinations may have different applications. For example, each of the dopamine substitutes listed above may contribute one or more different adhesion properties to the pDA coating, but certain dopamine substitutes may have particular utility in binding certain bacteria, fungi, and other microorganisms. Accordingly, some embodiments include (alongside or instead of dopamine) norepinephrine or other catecholamines.

In some embodiments, one or more pDA coatings 18 are coated over, around, or otherwise disposed on or in one or more substrates 20. Where the pDA coating is coated on a substrate, any suitable substrate can be used. For example, in some embodiments, the substrate includes one or more of: collection equipment (e.g., a test tube, a petri dish, a microscope slide, a sample vial, or any other collection equipment); a microfluidic device (e.g., a coating on the walls or any other suitable portion of any suitable microfluidic device); diagnostic equipment (e.g., for a medical or other diagnostic that requires separation of bacteria (or other microorganisms) from other entities or materials, or a medical or other diagnostic that requires concentration of bacteria (or other microorganisms)); magnetic particles (as discussed in more detail below); non-magnetic particles, such as beads (comprising any suitable type of silicate, glass, plastic, polymer, ceramic, natural material, synthetic material, or any other material), dust, inert particles, organic matter, or any other non-magnetic particles; an object or surface (glasses, plastics, metals, ceramics, semiconductors, or other objects or surfaces); a chromatography column; a carbon nanotube, carbon nanosphere, or other nanotube or nanosphere; graphite, graphene, or a similar surface; a bubble (e.g., a soap bubble, a gas bubble, or any other suitable type of bubble); a water filter (e.g., in a portable purification device, or in any other suitable filter); an air filter (or another gas filter); plumbing (e.g., in a pipe or drain, such as to purify the contents of the plumbing or to help assess the types of bacteria in the environment around the pipe or drain); walls; countertops; doorknobs; buttons; railings; drapery; cleaning materials (e.g., rags, wipes, mops, sponges, or any other suitable cleaning implements; or any other surfaces that may be useful for microorganism capture.

In some embodiments, at least some of the dopamine or dopamine substitute in the pDA coating 18 is polymerized. Although it can be polymerized in any suitable manner, some embodiments include one or more polymerizing agents or polymerizing environments. For example, some embodiments include polymerization in water, saline, or aqueous solutions (in some cases, containing dissolved agents (e.g., antibiotics, fluorescent molecules, etc.)), polar liquids, or another suitable solution in which dopamine (or an applicable dopamine substitute) will polymerize under the right conditions. In some cases, the substrate is placed into the solution along with the dopamine (or dopamine substitute) such that the dopamine (or dopamine substitute) polymerizes on the substrate, thereby forming a pDA coating.

In a similar vein, the substrate 20 can be coated in any suitable manner. For example, in some embodiments, the substrate is suspension coated, spray coated, dip coated, drip coated, roll coated, misted, cooked, powder coated, or coated in any other suitable fashion. By way of non-limiting illustration, methods for coating one or more magnetic particles with a pDA coating 18 are described in more detail in a later portion of this disclosure.

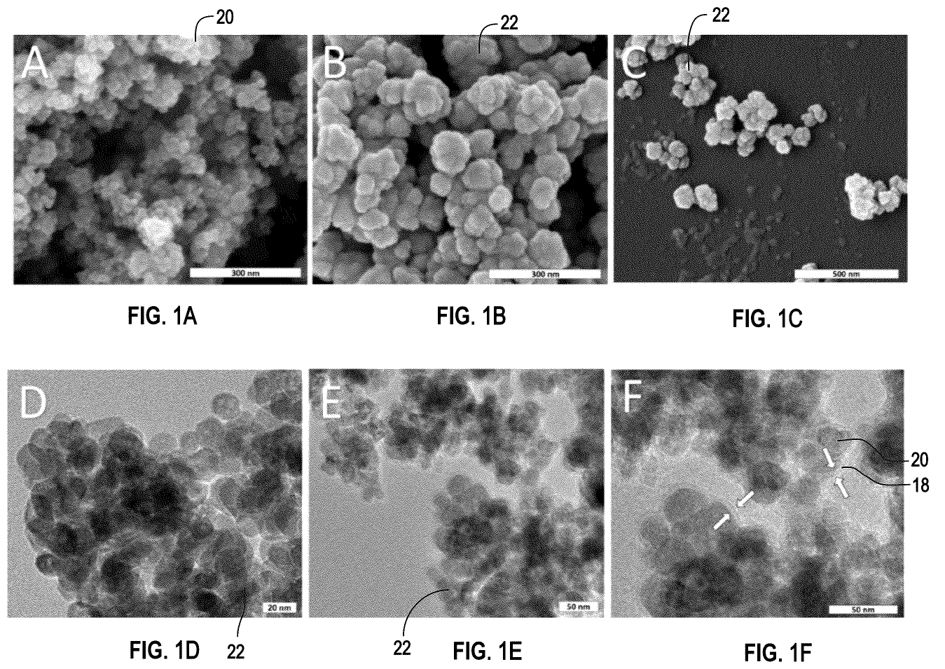

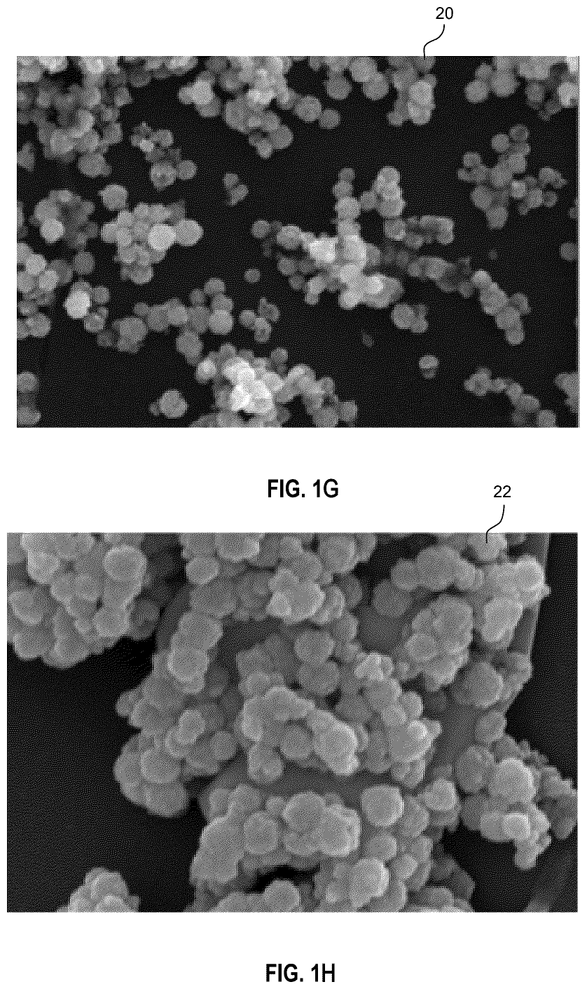

The pDA coating 18 can have any suitable thickness. For example, in some cases, the pDA coating is a relatively thin, conformal coating on the substrate 20, whereas in other cases, the pDA coating is relatively thick. In some cases, the pDA coating forms the substrate itself (e.g., beads or other materials formed of polydopamine or a polymerized dopamine substitute). That said, in some embodiments, the pDA coating has a thickness of between 1 nm and 1 mm, or within any subrange thereof (e.g., between 2 nm and 15 nm, between 3 nm and 10 nm, between 5 nm and 5 μm, or any other suitable subrange). In some embodiments, the pDA coating is quite thin, such as less than 15 nm, less than 10 nm, or less than 8 nm. By way of non-limiting illustration, some embodiments include a pDA coating having a thickness of 5.1±1.2 nm. In some cases, such a thin coating allows an underlying substrate to have a greater effect (for example, if the substrate is magnetic, more of the magnetism can, in some embodiments, be exerted than may be possible with a thicker coating. Indeed, in some embodiments, a thick coating may be polymerized to create not only more nominal surface area, but to create a rough surface texture that presents even more area for enhanced capture of the target species. By way of non-limiting illustration, FIG. 1F shows magnetic nano particles (MNPs) as a substrate 20, with arrows indicating a pDA coating 18 on the MNPs.

According to some embodiments, the pDA coating 18 is useful for binding one or more microorganisms 24. For example, in some cases, the pDA coating is coated on a substrate 20 that includes MNPs. The coated MNPs (or pDA-MNPs 22) can then be exposed to bacteria or other microorganisms (e.g., by exposing them to a fluid, such as a liquid or gas; by placing them on a solid, such as a countertop, a wipe with removable strands, or in any other suitable manner), allowing the pDA coating to bind the microorganisms. For example, the exposure can be through contact with a fluid containing the microorganisms, or with a solid (such as a solid surface of a solid fiber incorporated into a filter or wiping cloth). In some embodiments, the MNPs are then optionally extracted or removed from the fluid, solid, or other media, in any suitable manner. Indeed, in some cases, the MNPs are separated from a medium (e.g., a fluid or surface) using a magnet or by otherwise exploiting their magnetic properties, which (in some embodiments) causes the target microorganisms to be co-extracted.

Because pDA-MNPs 22 have many broad applications, a portion of the disclosure below focuses on pDA-MNPs. That said, the useful properties of the pDA coating 18 can be applied to many other substrates 20 (as discussed above). In this regard, with non-magnetic particles, separation (e.g., separation of the particles as bound to microorganisms from a liquid or solid medium) may need to be performed via centrifugation, filtering, optical trapping, optical tweezers, manual manipulation, or any other suitable processes, as pDA (generally speaking) is not itself magnetic, so the MNPs (or another magnetic substrate) would typically be required for magnetic separation. Accordingly, the discussion below relating to pDA-MNPs is not limited to use with a magnetic substrate (except where a magnetic substrate is clearly required, based on the context), but it also applies to non-magnetic particles and other substrates.

In accordance with some embodiments, the systems and methods described herein include one or more MNPs for capturing, retaining, extracting, purifying, concentrating, isolating, or characterizing one or more microorganisms. Where MNPs are included, any suitable MNPs can be used. For example, MNPs can include ferromagnetic particles, ferrimagnetic particles, paramagnetic particles, antiferromagnetic particles, superparamagnetic particles, superferromagnetic particles, diamagnetic particles, or any other suitable magnetic particles. Some suitable particles include compounds including iron, nickel, cobalt, neodymium, boron, and other compounds known to those in the art. As non-limiting examples, superparamagnetic particles of magnetite (Fe₃O₄) and maghemite (gamma-Fe₂O₃) can be produced in nano sizes. These and any other suitable magnetic nano particles can be used alone or in any combination. By way of non-limiting illustration, some embodiments include iron oxide MNPs formed from iron (III) chloride hexahydrate (FeCl₃-6H₂O). MNPs can be synthesized or prepared using any method currently known in the art or later developed, including co-precipitation, thermal decomposition, microemulsion, or flame spray pyrolysis. For example, illustrative methods of preparing MNPs are described in more detail below.

According to some embodiments, the MNPs (in some cases, when pDA-coated, and in other cases, while still uncoated) form into magnetic nanoclusters (MNCs), such as iron oxide nanoflowers (IONFs) or other nanoclusters, sometimes known as magnetic nanobeads. Generally speaking, many embodiments utilizing MNPs can also utilize MNCs. Accordingly, wherever suitable, discussion of MNPs herein also applies to MNCs and vice versa. Where MNC's are used, the MNCs can include any suitable number of MNPs, and can be any suitable size. Indeed, many MNCs contain between 2 and 200 MNPs, although there is no specific upper limit on the number of MNPs that could be included in an MNC. That said, some MNCs (in accordance with some embodiments) include at least 5, at least 10, at least 20, at least 30, at least 40, or at least 50 MNPs. Similarly, the size of an MNC can be any suitable size (and may not have any particular upper limit), but at least some MNCs (in accordance with some embodiments) have a diameter (or width or length) of between 5 nm and 10 mm, or any within subrange thereof (e.g., between 5 nm and 500 nm, approximately 250 nm±100 nm, or any other suitable size). In some cases, the MNCs have a crystallite structure (which can similarly be any suitable size, but in some cases is approximately 18 nm±8 nm).

In some cases, crystallites in an MNC behave not as individual magnetic units, but rather function together as a whole cluster (which again can have any suitable size, such as approximately 120±20 nm) that operates as a magnetic unit. In some cases, this allows for quicker microorganism extraction.

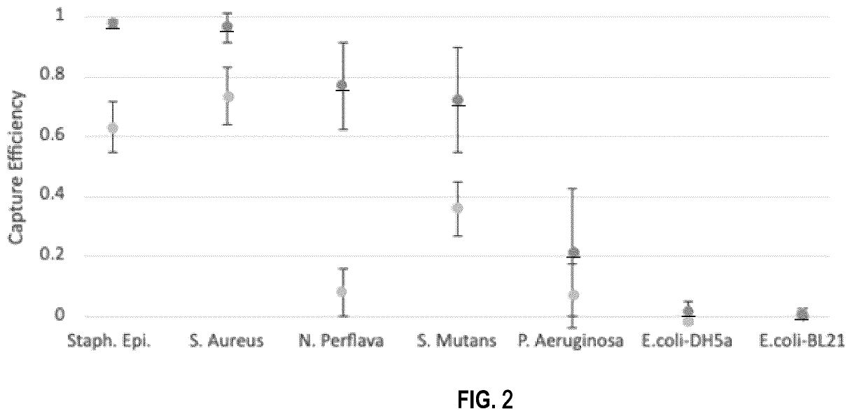

According to some embodiments, the pDA coating 18 is configured to increase the capture efficiency (CE) of the MNPs (or other substrates 20). For example, some embodiments increase the CE with respect to at least one microorganism 24 by at least 0.05, 0.1, 0.15, 0.2, 0.3, 0.4, 0.5, 0.6, 0.7, 0.8, or 0.9 (with 1 being full capture and 0 being no capture), based on suspension optical density (as described in more detail in a later portion of this disclosure). For example, some embodiments increase the CE with respect to one strain of bacteria by at least 0.2, and the CE with respect to another strain by at least 0.5. Measured another way, some embodiments of pDA coated MNPs have a CE that is greater than a CE of naked MNPs by over 50%, such as by 75%, 100%, 150%, 200%, 300%, 500%, 800%, or 1,000% greater (or even more, in cases where the MNP alone has very little CE and the pDA-coated MNP has very high CE). FIG. 2 shows some CE data (in accordance with Example 3 below), which shows marked improvements in CE of pDA-coated MNPs vs naked MNPs.

In some embodiments, the pDA-MNPs 22 (or other pDA-coated substrates 20) are configured to target (e.g., bind) specific bacteria or other microorganisms 24. For example, some embodiments are configured to target: gram-positive bacteria, certain types of gram-positive bacteria (e.g., gram-positive cocci, rods, or other classifications), gram-negative bacteria, certain types of gram-negative bacteria (e.g., gram-negative cocci, rods, or other classifications), Listeria subsp., Bacillus subsp., Staphylococcus subsp., Streptococcus subsp., fungi, algae, protozoa, archaea, viruses, or any other broad or specific target microorganisms. Indeed, some embodiments may be particularly useful for separating certain bacteria (or other microorganisms) from a biological milieu without co-extracting (or harming) other species in the biological milieu.

Relatedly, some embodiments of the pDA-MNPs 22 (or other pDA-coated substrates 20) are configured to specifically exclude certain types of microorganisms 24. For example, some embodiments do not adhere to (or have very low adherence to) gram-negative rod bacteria, E. coli, P. aeruginosa, or other specific strains of bacteria or other microorganisms. For example, in some cases, CE of one or more target organisms is greater than CE of one or more excluded organisms by at least 50%, 100%, 200%, 250%, 500%, 750%, 1000%, or even greater!

In some cases, one or more additional constituents used to target (or prevent the coating from targeting) certain bacteria or other microorganisms 24. For example, some embodiments of the pDA-MNPs 22 (e.g., without additional constituents) target many (if not all) gram-positive bacteria and some (but not all) gram-negative bacteria. Accordingly, some embodiments include one or more additional constituents—which can include anything that causes the pDA coating to interact with additional microorganisms—that prevent the pDA coating from interacting with one or more microorganisms that it normally would interact with, or otherwise affect the binding or other properties of, the pDA coating 18.

Relatedly, some embodiments of the pDA-MNPs 22 include one or more additional constituents to increase (or alter) the CE of the pDA-MNPs. For example, some embodiments include one or more binders configured to bind or more microorganisms 24, such as one or more antibodies, aptamers, lectins, polypeptides, polynucleotides, other complex biomolecules, ionic liquids (or other liquids), or any other suitable material that is configured to bind (alone or in connection with one or more other constituents) one or more microorganisms or parts thereof. As non-limiting examples, some embodiments of the pDA-MNPs include one or more of: vancomycin, imidazole groups, allantoin, penicillin, cefepime, teicoplanin, streptomycin, tetracycline, gentamicin, erythromycin, chloramphenicol, ciprofloxacin, rifampin, daptomycin, linezolid, colistin, bacitracin, clindamycin, amoxicillin, cephalexin, meropenem, imipenem, metronidazole, nitrofurantoin, fosfomycin, polymyxin B, other polymyxins, azithromycin, trimethoprim, sulfamethoxazole, mupirocin, gramicidin, amphotericin B, nystatin, fluconazole, itraconazole, caspofungin, micafungin, anidulafungin, defensins, cathelicidins, lysozyme, lactoferrin, LL-37, human beta-defensin 2 (hBD-2), human beta-defensin 3 (hBD-3), mannose-binding lectin, concanavalin A, wheat germ agglutinin, ricin B chain, Ulex europaeus agglutinin, monoclonal antibodies, immunoglobulin G (IgG), immunoglobulin A (IgA), aptamers, bacteriophage, CRISPR-Cas constructs, silver nanoparticles, gold nanoparticles, zinc oxide nanoparticles, chitosan, graphene oxide, peptidoglycan recognition proteins, toll-like receptor ligands, phospholipase A2, bile salts, gallium compounds, chelating agents (e.g., EDTA), surfactants (e.g., SDS), quaternary ammonium compounds, biguanides (e.g., chlorhexidine), ethanol, isopropanol, hydrogen peroxide, iodine, sodium hypochlorite, peracetic acid, benzalkonium chloride, triclosan, hexachlorophene, essential oils (e.g., eugenol, thymol, or any other suitable essential oil or oils), antimicrobial peptides (e.g., magainins, cecropins, or any other such peptides), or synthetic peptidomimetics. For example, some embodiments are configured to capture gram-positive bacteria via the pDA coating, and additionally include one or more polymyxins, allantoin, or other constituents configured to capture gram-negative bacteria (therefore allowing for a broader scope of capture). Some embodiments include additional constituents configured to target gram-positive bacteria, while largely leaving gram-negative bacteria alone (therefore allowing for even greater selectivity).

Notwithstanding the foregoing, some embodiments do not include one or more additional binding agents (e.g., as listed in the previous paragraph). As some non-limiting examples, some embodiments do not include vancomycin, some embodiments do not include imidazole groups, and some embodiments do not include allantoin. Indeed, some embodiments are configured to bind microorganisms irrespective of any additional binding agent (e.g., the pDA coating on the MNPs together, without any additional constituents, is sufficient to effectuate the desired CE).

According to some embodiments, the pDA-MNPs 22 are not toxic to microorganisms 24 (or are not toxic to specific target microorganisms). Indeed, some embodiments are configured to bind bacteria without killing the bacteria (or other microorganisms) or hindering the ability for such bacteria (or other microorganisms) to grow, reproduce, and thrive. Accordingly, in such embodiments, further growth, characterization, and other processes that require living microorganisms are possible after extraction. That said, in some embodiments, the pDA-MNPs include one or more anti-microbial agents or lysing agents configured to kill or disable the microorganisms upon or after extraction. For example, in some embodiments, it is desired to extract DNA or other markers from the microorganisms (e.g., to assist in the identification of species or antibiotic resistance).

The pDA-MNPs 22 can be any suitable size. For example, some embodiments of the pDA-MNPs range from between 0.5 nm to 5 mm in size (e.g., diameter), or any subrange thereof, such as between 1 nm and 3 mm, 2 nm and 2 mm, 1 μm±999 nm, or any other suitable subrange. In some embodiments, at least some of the pDA-MNPs have a size of less than 1 mm. In some cases, at least some pDA-MNPs have a size of less than 1 μm. In some cases, at least some pDA-MNPs have a size of less than 100 nm, or less than 50 nm, 20 nm, 10 nm, or 5 nm. Indeed, in some cases, nanoscale pDA-MNPs may be particularly useful for bacterial capture (e.g., 25 nm±15 nm).

In accordance with some embodiments, the systems and methods described herein include one or more methods for capturing, extracting, purifying, concentrating, isolating, moving, and/or characterizing one or more microorganisms 24. In this regard, the described methods can include any suitable systems or compositions described above, incorporated in any suitable manner (e.g., in any suitable order, have one or more portions of the methods be repeated, have one or more portions of the method be performed in series or in parallel with one or more other portions of the method, have one or more portions be omitted, have one or more portions be substituted or changed, or otherwise have the methods be modified in any suitable manner). For example, some embodiments of the described methods include one or more MNPs, which (in some embodiments) are coated with one or more pDA coatings 18. Thus, some embodiments include one or more methods for forming pDA-coated MNPs 22.

According to some embodiments, the described methods include obtaining one or more constituents for forming or coating MNPs. The constituents can include any suitable constituents, as described above or elsewhere herein (e.g., any of the constituents discussed in connection with the methods below).

According to some embodiments, the described methods include preparing one or more MNPs. This can be done in any suitable manner, such as via co-precipitation, thermal decomposition, microemulsion, polyol reduction, etching, milling, seed-mediated synthesis, thermal precipitation, grinding, sputtering, or via any other suitable method. As an example, preparing the MNPs includes, in some embodiments, obtaining any suitable solvent and dissolving or mixing any suitable solutes or nucleating particles therein to form a solution or suspension configured to form MNPs. While any suitable solvent can be used (e.g., 1,2-hexadecanediol, or any other suitable solvent), in some embodiments, the solvent includes ethylene glycol. That said, additional solvents may include water, ethanol, methanol, isopropanol, acetone, acetonitrile, dimethyl sulfoxide (DMSO), dimethylformamide (DMF), tetrahydrofuran (THF), diethyl ether, ethyl acetate, hexane, toluene, chloroform, dichloromethane (DCM), benzene, pyridine, or any other suitable solvent (or combination of solvents).

Where a solvent is used for the MNP-forming solution, any suitable amount of the solvent can be used. For example, some embodiments use between 5 mL and 5 L of solvent (or any subrange thereof), although for very small preparations, less than 5 mL can be used, and for large mass-productions, more than 5 L can be used.

According to some embodiments, the MNP-forming solution includes an organic salt, such as sodium acetate, salts of organic acids (e.g., acetic acid, formic acid, propionic acid, butyric acid, citric acid, or any other suitable acid), lithium acetate, potassium acetate, cesium acetate, calcium acetate, or any other suitable organic salts. The sodium acetate or other organic salt (alone or in combination) can be added in any suitable amount, such as between 0.1 g/L and 100 g/L (or any other suitable amount), or any subrange thereof (e.g., between 1 g/L and 5 g/L, between 2 g/L and 3 g/L, approximately 2.5 g/L±0.3 g/L, or any other suitable subrange).

According to some embodiments, the described methods include adding one or more MNP precursors to the solution. Where a precursor is used, the precursor can include anything configured to form one or more MNPs when added to the MNP-forming solution, such as iron (III) chloride hexahydrate, anhydrous iron (III) chloride, other ferric salts (e.g., ferric bromides or any other ferric salts), or any other suitable precursor. The precursor can be added in any suitable amount, such as between such as between 0.1 g/L and 10 g/L, or any subrange thereof (e.g., between 1 g/L and 5 g/L, between 2 g/L and 3 g/L, approximately 2.55 g/L±0.3 g/L, or any other suitable subrange). Indeed, some embodiments include adding iron (III) chloride hexahydrate.

In some embodiments, the described methods optionally include heating, stirring, agitating, or otherwise affecting the solution for a suitable duration of time to allow the MNPs to form. For example, some embodiments include intermittent or continuous agitation (e.g., via manual stirring, magnetic stirring, shaking, mixing, vortexing, rotating, rocking, or otherwise agitating the solution). Some embodiments include heating (e.g., via an autoclave, hotplate, heater, or otherwise), such as to a temperature of between 100° C. and 400° C., or any subrange thereof (such as approximately 200° C.±10° C.). In some embodiments, a different heat may be useful, depending on the particular constituents involved, so some embodiments use a temperature of between 0° C. and 500° C., or any subrange thereof. In some embodiments, this is done for a duration of between 1 minute and 48 hours, or any subrange thereof (e.g., approximately 10 hours±2 hours), or for any other duration suitable for causing MNPs to form.

In some embodiments, the method includes separating the MNPs from the remainder of the solution (e.g., using a neodymium magnet, or any other suitable magnet or method—in some cases, after cooling), and in some embodiments, the method includes washing the MNPs (e.g., with deionized water, HCl, phosphate buffered saline (PBS), NaCl, or any other suitable washing agent or combination thereof) to remove excess solution. In some cases, multiple washes are performed.

According to some embodiments, the described methods include coating the MNPs or any other suitable substrate 20 (e.g., including any of the substrates listed above) with at least one of dopamine and a dopamine substitute. While this can be done in any suitable manner, some embodiments include coating the substrate in a pDA coating solution. In some cases, the methods include coating the MNPs with multiple pDA coatings 18 (which may be the same coatings, using the same constituents, or different coatings using different constituents). For example, in some cases, the methods include forming a first coating that includes dopamine, and a second coating that includes a dopamine substitute (or vice versa). In some cases, a single coating includes both dopamine and a dopamine substitute.

Once again, where a dopamine substitute is used, any suitable dopamine substitute can be included. That said, certain dopamine substitutes may have attributes that are useful for either more comprehensive or more selective microorganism capture. Additionally, in some embodiments, the pDA-coating solution includes one or more other constituents configured to alter the CE with respect to one or more microorganisms (in some cases, to increase the CE; in some cases, to decrease the CE; and in some cases, to increase the CE with respect to a first microorganism and to decrease the CE with respect to a second microorganism).

Like the MNP-forming solution, the pDA coating solution can include any suitable solvent. For example, the solvent can include water, aqueous buffers, alcohols, PBS, mixtures of the foregoing, or any other suitable solvent system. By way of non-limiting illustration, some embodiments use PBS as a solvent. In some cases, the solvent is at a particular pH (as may be conducive to pDA coating), such as between 6.5 and 10, or any suitable subrange thereof (e.g., approximately 8.5±0.5). In some cases, the pH required for a certain rate of polymerization depends on the specific dopamine or dopamine substitute used (generally between 6.5 and 10).

Some embodiments include adding dopamine, or a dopamine substitute (e.g., any of the alternatives, derivatives, or other dopamine substitutes as described herein) to the solution. The dopamine (or alternative/substitute) can be included in any suitable amount, such as between 0.05 g/L and 50 g/L, or any subrange thereof (e.g., between 1 g/L and 5 g/L, between 1.5 g/L and 3 g/L, approximately 2 g/L±0.5 g/L, or any other suitable subrange). In some cases, the dopamine (or dopamine substitute) and the MNPs are added in approximately equal amounts (by weight), in some cases ±5%, 10%, 15%, 20%, 25%, 100%, or more of either.

Some embodiments include adding one or more additional constituents, such as one or more binders to alter CE (e.g., antibodies, ligands, or any other constituents as discussed above). In some cases, the additional constituents are configured to become embedded in (or to otherwise become part of) the pDA coating, thereby forming an altered coating on the substrate having different characteristics. In some cases, the additional constituents are added to the pDA coating after the coating process has commenced or even after the formation of the coating is completed (e.g., the constituents are added before, during, and after polymerization, in various embodiments). By way of non-limiting illustration, some embodiments include one or more antibodies (or other constituents) configured to target gram-positive bacteria (e.g., vancomycin, penicillin, erythromycin, clindamycin, daptomycin, linezolid, rifampin, bacitracin, cephalexin, ampicillin, or any other constituent configured to bind gram-positive bacteria), thereby further increasing the selectivity for gram-positive bacteria binding. Some embodiments include one or more antibodies (or other constituents) configured to target gram-negative bacteria (e.g., ciprofloxacin, gentamicin, ceftriaxone, aztreonam, meropenem, colistin, piperacillin, cefepime, amikacin, tigecycline, or any other constituents configured to target gram-negative bacteria), thereby further increasing the scope of binding of the pDA-MNPs 22.

Some embodiments of the methods include adding the MNPs (or other substrate) to the pDA coating solution, in any suitable amount, such as between 0.05 g/L and 50 g/L, or any subrange thereof (e.g., between 1 g/L and 5 g/L, between 1.5 g/L and 3 g/L, approximately 2 g/L±0.5 g/L, or any other suitable subrange). Although various portions of the methods can be performed in any suitable order, some embodiments include adding the MNPs before or directly after adding the dopamine or dopamine substitute to the pDA coating solution to take full advantage of the polymerization process to achieve a desirable coating.

Some embodiments of the methods include leaving the MNPs (or other substrate) in the pDA coating solution for a suitable duration of time to allow a pDA coating of a desirable thickness (e.g., any of the thicknesses discussed above, or any other desirable thickness) to form. In some cases, this is a relatively short duration of time, as may be desired to form a relatively thin coating (however, a longer duration may be used where a thicker coating is desired). In some embodiments, the pDA coating is performed under intermittent or continuous agitation (e.g., continuous magnetic stirring or any other suitable agitation). This can be done for any suitable duration of time, such as for between 10 minutes and 40 hours, or any subrange thereof (e.g., 10 hours±7 hours or approximately 3 hours±1 hour).

In some embodiments, the coated MNPs or other substrate(s) 20 are separated from the pDA coating solution (e.g., magnetically, via centrifugation, or otherwise), washed (e.g., in any suitable washing medium, such as PBS), or resuspended (e.g., in PBS, in some cases having a different pH, such as approximately 7.4±0.2).

According to some embodiments, the described methods include using the coating 18 (in some cases, as coated on one or more pDA-MNPs 22 or other substrates 20) to capture one or more bacteria (or other microorganisms 24). In some cases, specific bacteria (or other microorganisms) are targeted. In this regard, some embodiments of the methods include exposing the (coated) substrate to a fluid (including a liquid or a gas), such as by submerging the substrate, spraying the substrate, exposing the substrate to air flow or atomized particles, dipping the substrate, integrating the substrate into a device configured to contact a fluid, placing droplets on the substrate, placing the substrate on a desired surface or otherwise exposing the substrate to a fluid, to ambient conditions, to a desired surface, or any other suitable location for collecting microorganisms. This can be done for any suitable duration of time. In some cases, a relatively high CE can be achieved over a short period of time (e.g., less than an hour, less than 30 minutes, less than 20 minutes, less than 15 minutes, less than 10 minutes, or even shorter durations, in some cases). In some embodiments, even a time period of mere seconds (or less) is sufficient to collect a sample (depending on the particular conditions in question, such as the substrate concentration or binding affinity of the target microorganisms).

In some embodiments, the method includes incorporating the coating 18 into one or more tools, such as one or more diagnostic tools, collection tools, pieces of scientific measurement equipment, microorganism analysis equipment, or any other suitable tools (e.g., as discussed above). Thus, in some cases, the method includes extracting and analyzing the microorganisms 24 using a single unitary device. In some embodiments, the coating is incorporated into one or more surfaces of a water filter, such as a water filter for a portable water purification device (or any other suitable filter). In some embodiments, the coating is incorporated into one or more drains (or similar features) to assess bacteria in certain locations, such as hospitals and food processing plants. In some cases, the coating is applied to any other suitable surface, article, or material (e.g., filter, wipes, clothes, counters, walls, floors, paper, swabs, liners, surface covers, or any other suitable surfaces or materials).

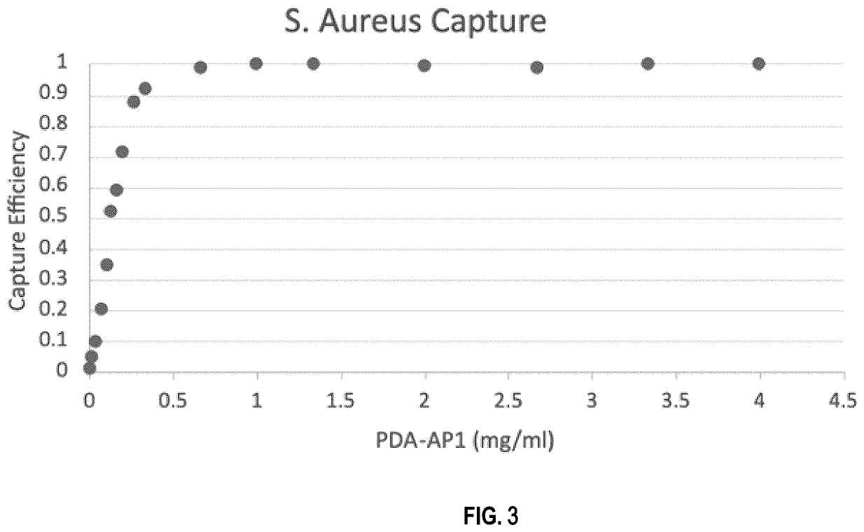

In some cases, the methods include partial microorganism 24 capture (e.g., to take a sample), whereas in some cases the methods are used for complete or near-complete microorganism capture (e.g., to filter one or more particular microorganisms out of a solution, treat an illness, purify a water source or food product, or otherwise remove microorganisms from a target source). (See e.g., FIG. 3.) In some embodiments, one or more of the described methods are used to capture dilute bacteria to provide a sample of concentrated bacteria. In some cases, the concentrated bacteria are further processed (e.g., cultured, characterized, isolated, purified, or otherwise processed). In some embodiments, the method is used for separation of specific target microorganisms from a biological milieu. For example, some bacteria are configured to produce valuable biomolecules, and it may be desirable to extract such bacteria from a particular environment without coextracting certain other bacteria or other microorganisms). On the other hand, in some embodiments, the pDA coating is configured to extract an undesirable bacteria (or other microorganism) from an environment without substantially affecting a microorganism that is desired to remain in place.

Generally speaking, the microorganisms 24 can be captured from any suitable medium. For example, in some embodiments, the microorganisms are extracted from a liquid medium, such as urine, plasma, serum, blood, cerebral-spinal fluid (CSF), puss, bile, stomach fluid, tears, sweat, water (e.g., fresh water, salt water, or other water from any source, such as taps, wells, rivers, lakes, oceans, water treatment systems, sewage, runoff, rain, or any other water sources), saline a solution, laboratory samples, chemicals, cleaning solutions, beverages (e.g., milk (including raw milk, pasteurized milk, milk derivatives or alternatives, and any other type of milk), fruit juices, vegetable juices brews, and any other beverages), meat broths, meat washings, fruit washings, vegetable washings, or any other liquids that may contain microorganisms. Additionally, in some embodiments, the microorganisms are captured from a solid surface or from any other suitable surface or material (e.g., from drapes in a hospital room, a countertop, dishes, food handling equipment, or any other suitable surface or material). According to some embodiments, the liquid includes a physiological fluid.

Where the liquid includes a physiological fluid, the extraction can be performed in vivo, in vitro, or a combination of in vivo and in vitro. Indeed, in some embodiments, the method includes exposing a pDA-coated substrate to physiological conditions inside a living (or deceased, such as the in case of performing an autopsy) patient (e.g., a human or animal patient). For example, the coated substrate can be exposed to a patient's blood, sweat, saliva, urine, discharge, or any other portion of the patient.

Some embodiments of the described methods include creating the fluid, surface, or other material containing or otherwise comprising the microorganisms 24. For example, if the microorganisms are present in soil, a solid object, or another non-fluid medium, the method can include dissolving, atomizing, soaking, rinsing, washing, percolating, treating, heating, cooling, or otherwise affecting the material in question to obtain a liquid or gaseous solution containing the target microorganisms. For example, in some embodiments, microorganisms are extracted from a surface or a solid medium. While this can be done in any suitable manner, some embodiments include dissolving the solid medium in a liquid medium (e.g., in any suitable solvent, such as water, ethanol, methanol, isopropanol, acetone, acetonitrile, dimethyl sulfoxide (DMSO), dimethylformamide (DMF), tetrahydrofuran (THF), diethyl ether, ethyl acetate, hexane, toluene, chloroform, dichloromethane (DCM), benzene, pyridine, or any other suitable solvent), and then extracting the microorganisms from the resulting solution. In some cases, a solvent that is not lethal to bacteria (or other microorganisms) is used. In some other embodiments, the pDA coating is added directly to a desired surface (e.g., a card, a strip of material, a bandage, a counter, a sticker, a slide, a piece of film, a piece of fabric, a button, and/or any other desired surface or material).

As an example of how a substrate 20 can be used to extract microorganisms 24 from a fluid or any other material, some embodiments include adding pDA-MNPs 22 (or another pDA-coated substrate) to the solution in question. In this regard, the pDA-MNPs (or another pDA-coated substrate can be collected in any suitable manner (e.g., via washing, rinsing, extracting pDA-coated fibers from a cloth, wiping up pDA-MNPs from a surface, using a magnet to collect the pDA-MNPs, or the pDA-MNPs are collected in any other suitable manner.

In some cases, once the pDA-MNPs are collected and added to a solution, the solution is agitated. In some embodiments, the pDA-MNPs (or other substrate) are then separated from the fluid (magnetically or via centrifugation, filtration, sedimentation, or by any other suitable means). In some embodiments, the separation process is performed (or is capable of being performed) in a short amount of time, such as in less than 5 hours, less than 15 minutes, less than 10 minutes, less than 5 minutes, less than 1 minute, less than 30 seconds, less than 10 seconds, or less than 5 seconds (or in any other suitable amount of time). Indeed, in some embodiments, the extraction and separation of the microorganisms from the target medium is capable of being performed under the method within about 12 minutes (±3 minutes).

In some embodiments, the methods include evaluating, testing, characterizing, or otherwise further processing the microorganisms 24. This can include processing in any suitable manner. For example, some embodiments include genetic characterization, such as via polymerase chain reaction (PCR) processing and genotyping (or any other suitable process). Some embodiments include phenotyping, such as phenotypic characterization via microfluidic analysis (or any other suitable process). Indeed, in some cases, incorporating the pDA coating into pipette tips, vials, microwells, microtubes, or other articles used in microorganism analysis can allow for small volumes of bacteria or other microorganisms to be analyzed without the need for time-consuming culture growth. In some embodiments, the methods include killing, deactivating, or lysing the microorganisms concurrently with or after extraction (e.g., for DNA extraction, characterization, or other processing). Accordingly, some embodiments include applying an antimicrobial or lysing agent after extraction, and some embodiments include forming a pDA coating that includes an antimicrobial or lysing agent.

The described systems and methods can be modified in any suitable manner. For example, some embodiments include (e.g., on the substrate, included in the coating, as part of a wash or treatment, or in any other suitable manner) antibiotics or any other suitable constituents configured to kill or deactivate microorganisms 24.

In addition to the aforementioned features, the described systems and methods can include any other suitable feature. For example, some embodiments allow for very rapid analytics compared with traditional methods. Additionally, some embodiments allow for a high degree of selectivity, which can be customized using additional binding constituents.

### EXAMPLES

### Reagents and Materials

For the following examples, anhydrous sodium acetate was purchased from JT Baker (Phillipsburg, NJ, USA). Iron (III) chloride hexahydrate, ethylene glycol, sodium chloride, and monosodium phosphate hexahydrate were purchased from Fisher Scientific (Waltham, MA, USA). Potassium chloride was purchased from Spectrum Chemical Mfg. Corp. (New Brunswick, NJ, USA). Monopotassium phosphate was purchased from Millipore Sigma (Burlington, MA, USA). 3-Hydroxytyramine hydrochloride (dopamine) was purchased from TGI (Portland, OR, USA). Ultrathin carbon film on a holey/lacey film, 400 mesh, and Cu grids were purchased from Ted Pella, Inc. (Redding, CA, USA). Apple juice was purchased from a local market (Minute Maid™, Coca-Cola Company, Atlanta, GA, USA).

### Example 1—Preparation and Coating of MNPs

To make MNPs, 50.0 mg of anhydrous sodium acetate was dissolved in 20 mL of ethylene glycol for 10 minutes with continuous magnetic stirring by stir bar. Then iron (III) chloride hexahydrate (54 mg) was added to the solution and stirred for 15 minutes. The solution was then sealed into a 100-mL Teflon-lined autoclave (AS-017, Mayiya™, Amazon™, Seattle, USA) and heated at 200° C. for 10 hours.

The autoclave was cooled to room temperature with convective air and the MNPs were separated with a neodymium magnet and washed three times with 20 mL of deionized water. About 100 mg of the nanoparticles were added to 40 mL of HCl (1 M) and magnetically stirred for one hour. The nanoparticles were magnetically separated and washed three times with 20 mL of PBS (pH=7.4). The MNPs were then stored in PBS (pH=7.4) at room temperature.

To coat the MNPs, 50.0 mg of MNPs were added to 25.0 mL of PBS at pH=8.50 and placed under continuous magnetic stirring with a stir bar. Then, 50.0 mg of dopamine were added to the solution and stirred at room temperature (RT) for three hours. The coated MNPs were then separated with a neodymium magnet under the foil, washed three times with 20 mL PBS (pH=7.4) and resuspended and stored in PBS (pH=7.4).

To determine the concentration of MNPs (coated or uncoated) in a PBS suspension, 100 μL of rigorously vortexed suspension was pipetted onto dried and tared aluminum foil. Next the MNPs were washed with deionized (DI) water to remove PBS while holding the MNPs in place on the foil with a neodymium magnet. The sample was dried for 20 minutes on a hotplate set to 120° C., weighed, and then the mass of the MNPs was calculated. For each production batch, three or four aliquots were measured to calculate the MNP mass per volume of suspension. Then, the MNP concentration was adjusted to 13.3 mg/mL in PBS by adding or removing PBS. A standard 13.3 mg/mL suspension of MNPs in PBS was initially used in the examples involving bacteria below.

The above process was successful in producing MNPs-particularly in a cluster morphology (MNCs). FIGS. 1A-1H show SEM and TEM images of MNPs 20 (in some cases bare, and in some cases coated (pDA-MNPs 22)). FIG. 1A (SEM, bare MNPs) shows MNCs that were measured to be 120±40 nm in diameter (n=43). The MNC's were formed from spherical or generally spherical MNPs that have approximately a 19.5±3.0 nm (n=32) diameter, based on TEM (see FIG. 1D). The TEM measurement of MNPs 20 have a diameter of 18.7±4.3 nm (n=21) (see FIG. 1D (TEM, bare MNPs), which shows some Moiré patterns where two primary particles overlap, suggesting that there is (in some embodiments) crystalline structure in the primary particles). Some individual primary MNPs are seen in FIG. 1D, having a diameter of 18.7±4.3 nm (n=21) as viewed by TEM. The size measurements using SEM and TEM were not statistically different in this example (p=0.463), so for this example, a value of 19 nm is used as the size of a primary particle. For the average size of an MNC, 120 nm is used for this example. FIG. 1B (SEM, pDA-MNPs) shows clusters of MNPs coated with pDA and a thin coating of sputtered metal that was deposited to enable better SEM imaging. The metal layer was carefully controlled to be 10.0 nm, so the remaining difference in diameter is attributed to the thickness of the pDA layer. The size of the clusters in FIG. 1B is 145 nm±20 nm (n=14). These clusters are larger than those in FIG. 1A, supporting the proposition that there is a pDA layer and a metal layer on the cluster. Moreover, FIGS. 1E and 1F (TEM, pDA-MNPs) show the pDA coating 18, which is estimated (in this example) to be 5.1±1.2 nm (n=15).

To calculate how many primary MNPs were in 1 g of MNPs, the particles were assumed to have the density of magnetite (e.g., about 5.17 g/cc) and have a diameter of about 19 nm. The mass of a primary particle was estimated to be about 1.9×10⁻¹⁷ g. To calculate the number of MNPs in a cluster, an average cluster diameter of about 120 nm was assumed, and it was assumed that a cluster is made of solid magnetite spheres with a random packing factor of about 0.64. The mass of a cluster was estimated to be about 3.0×10⁻¹⁵ g, and thus an average cluster in this example contained about 160 MNPs.

### Example 2—Characterization of Magnetic Nanoparticles

MNP samples were washed three times with 10 mL of DI water and then diluted to 0.4 mg/mL in distilled water for both transition electron microscopy (TEM) and scanning electron microscopy (SEM) procedures.

For SEM visualization, 10 μL of suspension were transferred to a silicon wafer and dried in a desiccator for 24 hrs. To produce coated samples, Au/Pd was sputtered on the MNPs for about 60 seconds to form 10.0 nm of Au/Pd using a Q150T-ES thin-film coater (QUORUM, Sacramento, CA, USA). SEM imaging was performed using an Apreo™ C Low-Vacuum SEM (ThermoScientific, Waltham, MA, USA) operated at an accelerating voltage of 5-15 mV.

For TEM imaging, 8.0 μL of suspension were transferred to an ultra-thin carbon film on a holey/lacey film, 400 mesh, Cu grid, and dried in a desiccator for 24 hrs. TEM imaging was preformed using a Tecnai™ TF-20 TEM (FEI, Hillsboro, OR, USA).

The images were processed with ImageJ™ software (NIH, Bethesda, MD, USA) to obtain the diameters of particles.

X-ray photoelectron spectroscopy (XPS) was done as follows. Double-sided tape was attached to a silica wafer. A dry sample of MNPs was placed on the upper side of the tape and XPS spectra was obtained with a K-Alpha™ X-ray Photoelectron Spectrometer (ThermoScientific, Waltham, MA, USA). The data was processed using CasaXPS™ software (Casa Software Ltd, Teignmouth, Devon, UK).

X-ray diffraction data for MNPs and pDA-MNPs were collected using a Panalytical X'Pert Pro™ MPD with an X'Celerator™ detector, a Cu X-ray source with a Ge monochromator and Kα1 wavelength (λ=1.5406 Å). The beam was conditioned using fixed divergence and antiscatter slits (0.25°), Soller slits (0.04 rad), and a mask (10 mm). The PHD (pulse height detection) lower and upper levels of the detector were adjusted to 55% and 80%, respectively to avoid Fe fluorescence. The sample was loaded into a zero-background holder and scanned between 14° and 124° (2θ) with a step size of 0.0084°/step and a counting time of 350 s/step. NIST LaB6 line position and line shape standard (660b) data were collected over the same range of 20 using the same instrument configuration. The XRD data obtained were matched to a magnetite pattern (PDF #04-015-9120) in the ICDD Web PDF-4+ database. The MNPs, PDA-MNPs and the LaB6 standard reference material were profile fit with the Highscore Plus™ software using Pearson VII profile functions and polynomial backgrounds. The Gaussian and Lorentzian coefficients from the profile fit of the LaB6 were used to estimate and subtract the background from the instrument to the peak width in the MNPs and pDA-MNPs.

Dynamic light scattering (DLS) and zeta potentials were measured at 25° C. in a Zetasizer™ Nano Zs (Malvern Instruments, Malvern, Worcestershire, UK). MNPs or pDA-MNPs 22 were diluted to 0.1 mg/mL in PBS for the analysis.

The mass of coating on the MNPs was determined as follows. Bare or pDA-MNPs 22 were dried in an oven at 70° C. for 24 hours. A sample was transferred to a ceramic crucible, weighed, and placed in an oven preheated to 1000° C. After 3 hours, the crucible was cooled, and weighed again. The crucible was placed back into the oven for three hours, cooled, and weighed again. This process was repeated one more time. A final change in mass was calculated.

Magnetometry data was collected on the MNPs and pDA-MNPs 22 via vibrating sample magnetometry (VSM) using a Physical Properties Measurement System (Quantum Design, San Diego, USA) that includes a superconducting magnet producing a magnetic field up to 9 T, and a cryostat allowing temperature variations in the range of 10 K to 400 K. To collect zero field cooling (ZFC) data, the sample was cooled down to 20 K and then field was applied, and data was collected while warming up the sample. In contrast, field cooling (FC) data after first applying the magnetic field and then cooling the sample to 20 K, and the data was collected while warming up the sample.

In connection with this Example 2, the academic article entitled Bacterial Binding to Polydopamine-Coated Magnetic Nanoparticles, by Bowen J. Houser et al., ACS Applied Materials & Interfaces 2024 16 (43), 58226-58240, DOI: 10.1021/acsami.4c11169 (hereinafter, “the Houser Article”) provides some additional information. Accordingly, Sections 3.2 and 3.3 of the Houser Article are hereby incorporated into this example by reference, including the supporting information and figures discussed in those sections.

### Example 3—Bacterial Preparation

At least eight strains of bacteria were studied: Staphylococcus aureus (S. aureus, strain ATCC #12600), Staphylococcus epidermidis (S. epidermidis, strain RP62A), Streptococcus mutans (S. mutans, strain ATCC #700610), Neisseria perflava (N. perflava, strain ATCC #14799), Pseudomonas aeruginosa (P. aeruginosa, strain PAO1), and 3 strains of Escherichia coli (E. coli: DH5α, strain BL21 containing a plasmid coding for a green fluorescent protein (GFP), and ATCC strain 25922). In this example, a correlation for each species was done between optical density at 600 nm (OD₆₀₀) and bacterial concentration in colony forming units (CFU) per mL, as measured by plate counting.

Each of the above strains was streaked from frozen culture onto an appropriate plate, grown at 37° C., and a single colony was used to inoculate 20 mL of appropriate growth media. After overnight culture, a 500 μL aliquot was used to inoculate 50 mL of growth media in a shaker flask. The flask was shaken on an orbital shaker at 120 rpm and incubated at 37° C. until the culture was in the logarithmic phase of growth. See Table 1 (below) for growth times used to attain log-phase phenotype.

After the appropriate growth time, the suspension was harvested and centrifuged 10 minutes at 488 g to form a pellet. The pellet was washed twice by resuspending the bacteria in about 2-3 mL PBS, followed by vortexing and then centrifuging 10 minutes at 488 g. After additional resuspension of the pellet, the bacterial suspension was diluted with PBS to give an optical density (OD) reading between 0.5 and 1.2, as measured on a Cary 60 UV/vis spectrometer at 600 nm (OD₆₀₀). The OD is defined as:

$$OD = - {log}_{10} \frac{{I}_{°}}{{I}_{i}}$$

where Iₒ is the intensity of light exiting out of the sample, and Iᵢ is the intensity of light entering into the sample.

The change in optical density is generally considered a measurement of adhesive capture (the optical density of bacterial suspensions has long been attributed to the concentration of bacteria). This method of measuring bacterial capture has several advantages. For example, when doing plate counts of colony-forming units, some bacteria may remain associated with each other and produce a single colony, thus creating an undercount of the true concentration, whereas this is not an issue with optical density analyses.

TABLE 1

| | | | Time to | |
|---|---|---|---|---|
| | Plate | Culture | logarithmic | |
| Species | media | media | culture | Notes |
| S. aureus | NA¹ | NB¹ | 4 hr | |
| S. epidermidis | TSA² | TSB² | 4 hr | |
| S. mutans | BHA³ | BHI³ | 12 hr | Grown in 5% |
| | | | | CO₂ in air |
| N. perflava | MHA⁴ | MHB⁴ | 12 hr | |
| P. aeruginosa | LBA⁵ | LBB⁵ | 4 hr | |
| E. coli DH5 α | NA¹ | NB¹ | 4 hr | |
| E. coli BL21 | NA¹ | NB¹ | 4 hr | |

¹Nutrient agar and broth
²Tryptic soy agar and broth
³Brain heart infusion agar and broth
⁴Mueller Hinton agar and broth
⁵Luria Bertani agar and broth

### Example 4—Measurement of CE of Bacteria by MNPs

The bacterial suspensions (e.g., as prepared in Example 3) were diluted to around 3×10⁸ CFU/mL as estimated by OD₆₀₀. One mL of suspension was added to each of three microcentrifuge tubes (1.5 mL tubes) which were assigned as: (1) Control, (2) Naked MNPs, and (3) pDA MNPs. Next, 300 μL of PBS were added to the control tube, 300 μL (taken from a 13.3 mg/mL prepared suspension) of vortexed bare MNPs were added to the second tube, and 300 μL (13.3 mg/mL) of vortexed pDA-coated MNPs were added to the third tube, which gives a ratio of about 700,000 MNPs/CFU. Rigorous vortexing of MNPs was done just before adding them to a culture suspension to break up loose aggregates of MNP clusters. During 10 minutes of contact time, the MNPs were kept in suspension by flipping the microfuge tubes upside down at least every two minutes (this precluded settling of the MNPs in the sample).

After 10 minutes, a hand-held neodymium magnet was moved around the outside of the tube to collect most of the MNPs into one spot on the inside wall of the tube. Then each tube was placed in a magnetic rack for two minutes, which has a single neodymium magnet in close proximity (˜1 mm) to the outer surface of the microfuge tube. Next, while the tube was still in the magnetic holder, 1 mL of aliquot was carefully removed by pipet without disturbing the MNPs collected by the magnet on the side of the tube. Suspension turbidity (OD₆₀₀) was measured on the decanted liquid using the UV/vis spectrometer.

The control tube measurement represents the expected turbidity of the bacteria in the other two tubes before they were contacted with MNPs. The turbidity in the other two tubes represents the concentration of bacteria that were not captured by the MNPs.

From the remaining 0.3 mL of liquid, 10 μL were removed and serially diluted for plate counting. Plates were incubated for 48 hours and counted by hand.

The CE of the MNPs is defined as the fraction of bacteria that adhered sufficiently to the MNPs that were pulled from the liquid by application of the magnet. Mathematically, it is defined as:

$$CE = 1 - \frac{{OD}_{6 \; 00 , after}}{{OD}_{6 \; 00 , before}}$$

where OD_{600,before} and OD_{600,after} are the OD₆₀₀ values of the liquid phase of the sample before and after (respectively) exposure to the tube's contents and subsequent magnetic collection.

CE quantitates the fraction of bacteria pulled out of suspension by MNPs. It also reflects how strongly a bacterium is attached to a MNP and remains connected during the magnetic “dragging” of MNPs toward the external magnet, despite the drag force being relatively strong.

FIG. 2 shows, in accordance with some embodiments, the adhesive CE of the particles towards various strains of bacteria as measured by depletion of suspended bacteria as the MNPs were extracted from the suspension. As defined above, the adhesive capture is the fraction of suspended bacteria that was removed from the suspension by incubation with MNPs followed by magnetic removal of the MNPs. Adhesive capture was measured by the reduction in optical density of the original and extracted bacterial suspensions, confirmed with plate counts. In all cases, the magnetic particles were in contact with the bacteria suspension for 10 minutes before being pulled to one side of the container by an external magnet for at least two minutes.

FIG. 2 compares the adhesive capture of the bacterial strains, ranked from most to least captured, based on suspension optical density at 600 nm. In this example (e.g., based on the particular embodiment produced by the described protocol), it is apparent that Gram-positive rods and cocci, represented by Staphylococcus and Streptococcus species were extracted more efficiently from the suspension by the pDA-coated MNPs than the P. aeruginosa and E. coli were. It is worth noting that this is not strictly a gram-negative versus gram-positive observation, as N. perflava is a Gram-negative cocci, and it was extracted by the pDA-coated MNPs better than extraction of the S. mutans. Accordingly, various embodiments of the systems and methods described herein are useful for targeting specific strains of bacteria and other microorganisms. Indeed, some embodiments can be used in connection with other systems and methods for extracting, purifying, characterizing, and otherwise dealing with microorganisms for a calculated result (e.g., filtering out gram-negative bacteria, and then extracting only such gram-negative bacteria as are efficiently captured by a particular embodiment of pDA-coated MNP.

As an additional note about FIG. 2, the bacterial strain that most adhered to the pDA-coated MNPs (in accordance with the applicable embodiment) was S. epidermidis (CE=0.97+/−0.01), followed by S. aureus (CE=0.96+/−0.05) and N. perflava. The strains that least adhered to the applicable embodiments of the coated particles were P. aeruginosa and E. coli. It is also worth noting that S. epidermidis and S. aureus appeared to have excellent adhesion to the naked MNPs (in accordance with some embodiments). For example, S. epidermidis aureus had the highest CE of 0.73+/−0.10 (mean and standard deviation, n=4) by naked nanoparticles, followed by S. aureus epidermidis at and 0.63+/−0.08, as measured by optical density changes.

Moreover, the residual black material, which included bacteria associated with pDA-coated MNPs, was diluted in growth media and its growth curve was measured at 37° C. It is noteworthy that for all species, the lag times and log phase growth rates of bacteria associated with beads were not different from those of bacteria that were never in contact with MNPs. This suggests that these pDA-coated MNPs are not toxic to this set of bacteria as evaluated by growth curves.

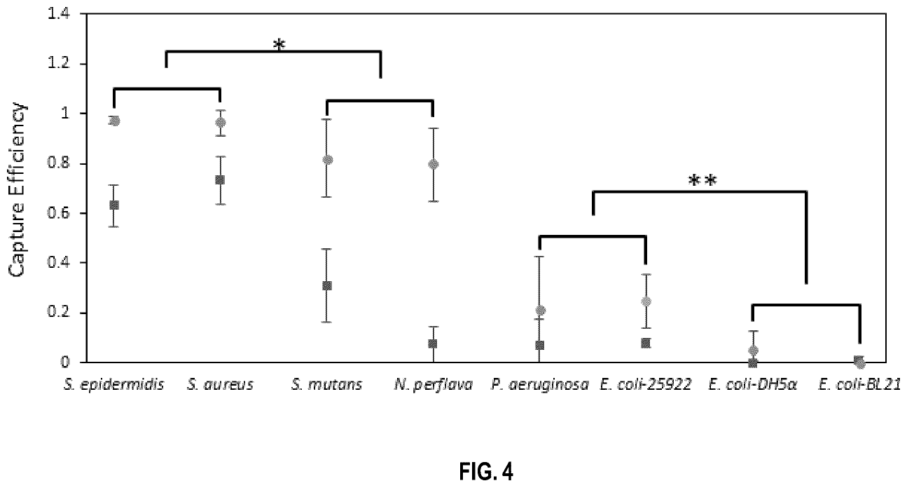

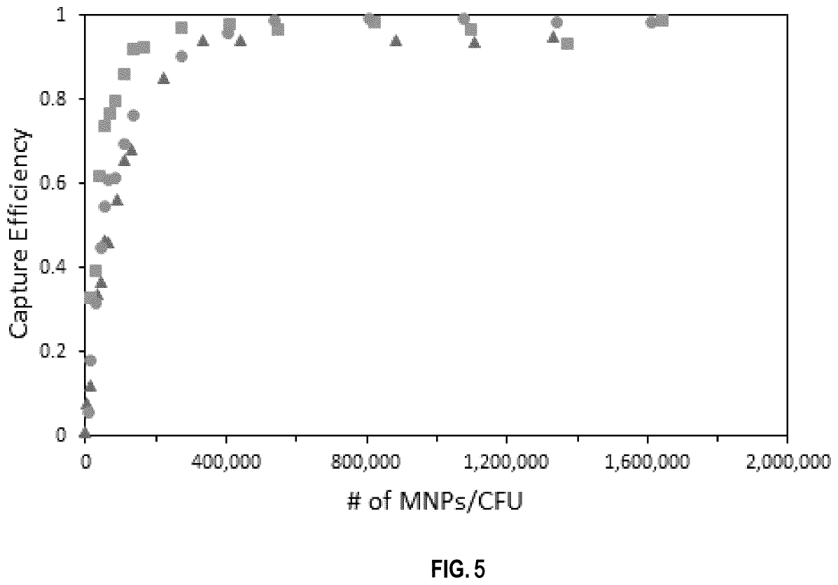

FIG. 4 shows the ranked CE of the 8 strains of bacteria as measured by depletion from a bacterial suspension after 10 minutes of exposure to pDA-MNPs. Based on the data in this example (for the related embodiments), gram-positive cocci, represented by Staphylococcus and Streptococcus species, are extracted much more efficiently from the suspension by the pDA-MNPs than are E. coli. This is not strictly a gram-negative or gram-positive phenomenon, as N. perflava is a gram-negative cocci, and yet it was extracted by the pDA-coated MNPs with a fairly high CE of 0.80±0.15 (n=6). The gram-positive cocci, S. mutans, showed a similarly high CE (0.82±0.16, n=8), and this value was not statistically different from the N. perflava value (p=0.77).

In contrast, the gram-negative species P. aeruginosa and E. coli had very low CE by the PDA-coated MNPs (in accordance with the related embodiments). As seen in FIG. 4, P. aeruginosa (strain PAO1) and E. coli strain 25922 were captured at efficiencies between 0.21 and 0.25, whereas the other two E. coli strains showed nearly zero adhesion to these PDA-coated MNPs under these conditions (e.g., CE=0.001±0.01 for E. coli BL21 strain).

Again, some bacteria appeared to adhere well to the bare MNPs (in accordance with the relevant embodiments). For example, S. aureus had the highest CE of 0.73+/−0.10 (n=4) by bare nanoparticles, followed by S. epidermidis at 0.63+/−0.08 (n=6), as measured by turbidity changes. Yet the N. perflava and S. mutans had low to very low CE by bare MNPs (0.07±0.07, n=6 and 0.31±0.14, n=8, respectively). Adhesion of P. aeruginosa and E. coli to bare MNPs was also low.

In this particular example, in the addition of the pDA coating to the MNPs did not decrease the adhesion as measured by CE. Similar data measured by plate counts are shown in the Supplemental Materials referenced in Section 3.4 and figure S2 of the Houser Article, which Section and figure are each incorporated into this example by reference.

### Example 5—Measure of S. aureus as a Function of MNP Concentration

The CE was measured with respect to the capture of S. aureus across decreasing ratios of pDA-coated MNPs to bacteria, with the results shown by FIG. 3. In particular, FIG. 3 shows the CE of S. aureus as a function of the amount (in mg) of pDA-MNP beads per mL of bacterial suspension, which suspension had 1.2×10⁸ CFU S. aureus in PBS. Generally speaking, these results indicate that, for the applicable embodiments, CE was proportional to the amount of pDA-MNPs at low concentrations up to about 0.3 mL. At MNP amounts greater than 0.8 mL, the CE is nearly 1, indicating that there are sufficient numbers of MNPs to capture a very large percentage (e.g., 90%+, 95%+, 98%, 99%+, or even greater) of the bacteria.

### Example 6—Additional Saturation Experiments

To investigate the CE of the pDA-MNPs as a function of pDA-MNP concentration, bacterial capture experiments similar to those in the previous examples were performed, but with different initial pDA-MNP concentrations. Bacteria suspensions were cultured, washed, and resuspended in PBS as described above. One mL of a 1.2×10⁸ CFU/mL suspension was added to 1.5 mL-sized microcentrifuge tubes. Then, 300 μL pDA-MNP suspension was added to the microcentrifuge tubes at varying mass concentrations ranging from 0 to 13.3 mg/mL pDA-MNP, producing a particle ratio of 0 to 1,750,000 MNPs/CFU. The MNPs were kept in suspension for 10 minutes on a mechanical rotator that tumbled the samples end-over-end at 125 rpm. After the 10-minute exposure time, the tubes were placed on a magnetic holder with a neodymium magnet for two minutes. 1 mL of aliquot was decanted, and its OD₆₀₀ was measured. CE was calculated as described above.

At a constant exposure time of 10 minutes, CE increased with increasing ratio of MNPs to CFU until a certain point (which may be referred to as “saturation”), where additional MNPs per CFU do not substantially increase CE further. At approximately 400,000 MNPs/CFU, both S. epidermidis and S. aureus reached a maximum CE greater than 0.96; but S. mutans, which exhibited less effective binding to MNPs (see FIG. 4), only reached a maximum CE greater than 0.93 (at about the same value of 400,000 MNPs/CFU, after which point the CE no longer continued to increase with increasing value of MNPs/CFU).

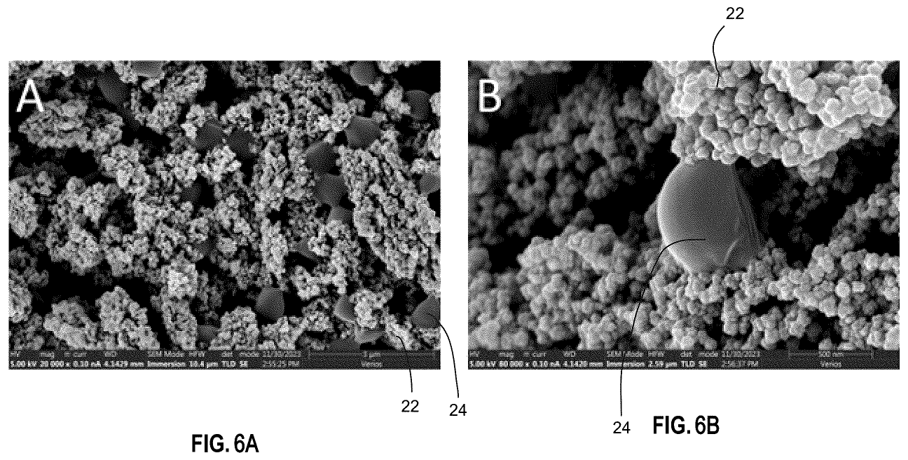

The black pDA-MNPs captured magnetically on the inside of the vial were examined by SEM. For those samples which were exposed to S. epidermidis, S. aureus, and S. mutans, bacteria were consistently observed to be embedded within the pDA-MNPs, as shown in FIGS. 6A-6B. It is noteworthy that the observed bacteria appeared to have bare patches that did not have adherent beads (otherwise they could not have been seen). In many cases the observable bacteria appeared to be deformed from the expected spheroidal (or oblate spheroidal) shape. The ratio of MNPs to bacteria (in FIGS. 6A-6B, and in accordance with this example) was about 27,000 MNP/CFU, which correlates to a CE of about 32%. More examples of pDA-MNPs attached to bacteria are found in figure S4 of the supplemental materials of the Houser Article, which figure is incorporated by reference into this example.

### Example 7—Kinetics Experiments

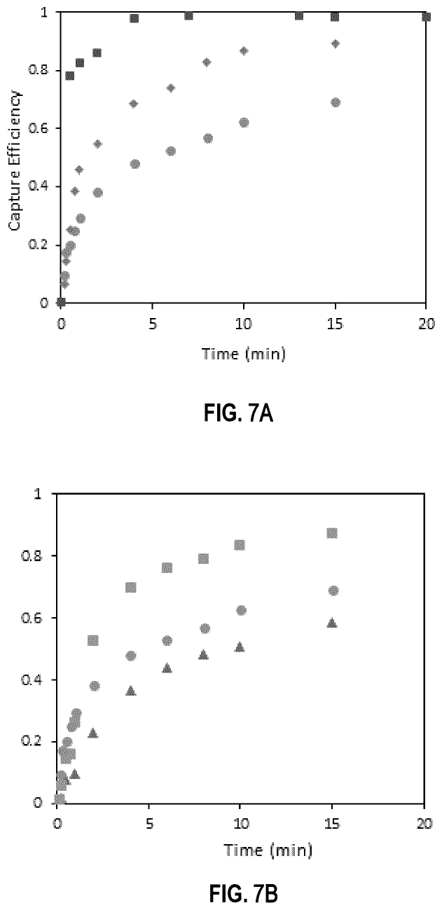

CE of the pDA-MNPs as a function of bacterial exposure time to pDA-MNPs was also investigated. Experiments were performed the same as in Example 6, except that instead of varying pDA-MNP concentration, exposure time was varied between 0 and 15 minutes. The pDA-MNP concentrations chosen for this example were generally lower than the MNP saturation concentration (400,000 MNPs/CFU or less), so that change in CE with respect to time could be investigated without overly rapid capture kinetics (an example of such rapid capture kinetics can be seen in the data of FIG. 7A for 400,000 MNPs/CFU of S. aureus). As exposure time increased, CE continued to increase until saturation was achieved. Since most of the MNP concentrations were below the saturation concentration for 10-minute exposure time in this example, it was not determined whether an even greater value of CE would be achieved at exposure times longer than 10 minutes. There appeared to be a dynamic (time dependent) interaction between MNP/CFU ratio and capture kinetics.

An initial evaluation of the data shown in FIG. 7A was done to estimate the initial capture rate, calculated as the slope of CE vs. time, at t=0. These initial rates were proportional to the MNP concentration, suggesting that for at least S. aureus and S. mutans, the rate of capture is first order with respect to MNP concentration. Portions of the supplemental materials of the Houser Article include additional information, including figure S5 and the accompanying descriptions, which are hereby incorporated by reference into this example.

### Example 8—Selective Capture Experiments

To investigate whether differential CE of individual bacterial strains by pDA-MNPs is retained in a mixture of bacterial strains, the following experiments were performed. A bacterial suspension composition of S. epidermidis and E. Coli-BL21 was chosen because these strains lie at the extremes of CE (based on the previous examples). The same bacterial capture experiment was performed as in Example 4, but instead of using 1 mL of a single strain in each microcentrifuge tube, 0.5 mL of 3×10⁸ CFU/mL S. epidermidis and 0.5 mL of 3×10⁸ CFU/mL E. Coli-BH21 were added to each tube.

After bacterial capture and magnetic collection, the decanted samples were serially diluted and plated on both eosin-methylene blue (EMB) agar and mannitol salt agar (MSA). E. coli grows on EMB but it does not grow on MSA, and S. epidermidis grows on MSA but it does not grow on EMB. We also confirmed that the E. coli used produced the same number of colonies on EMB agar as it does on nutrient agar, and the S. epidermidis used grew the same number of colonies on MSA as it does on TSA plates. The E. coli colonies were easy to distinguish from S. epidermidis by their expression of green fluorescent protein (GFP), so we were able to confirm species identity by plating the decanted mixture on each type of selective growth plate. From this data we calculated the CE from plate counts for each species i, CE_{pc,i}:

$${CE}_{p c , l} = \frac{{CFU}_{0 , i} - {CFU}_{i}}{{CFU}_{0 , i}} = 1 - \frac{{CFU}_{i}}{{CFU}_{0 , i}}$$

where CFU_{0,i} and CFUᵢ represents the number of CFU's of species i before introduction of MNPs and after magnetic separation, respectively.

The pDA-MNPs exhibited selectivity in preferentially capturing S. epidermidis from a mixture of S. epidermidis and E. coli-BL21. The capture efficiencies were 0.86±0.16 and 0.12±0.25 (mean±standard deviation), as shown in supplemental figure S6 of the Houser Article, which is hereby incorporated by reference (along with the accompanying description in the supplemental information in the Houser Article) into this example. This example includes larger standard deviations, at least in part due to quantitating CE with plate counts instead of OD₆₀₀.

While the mean S. epidermidis CE appeared slightly less than in a pure S. epidermidis suspension (compare to FIG. 4), it was not statistically different (p=0.21, n=7). Furthermore, it still greatly surpassed the CE of E. coli in the mixture (which was not statistically different than the OD₆₀₀ data of FIG. 4, p=0.40, n=8). Accordingly, CE persisted in mixed cultures, suggesting the potential of using pDA-MNPs for separation of bacterial species in mixtures. These results also demonstrated that the adhesive properties of pDA-MNPs to S. epidermidis were not significantly compromised by the presence of other non-adhesive bacteria.

### Example 9—Capture from Apple Juice

To demonstrate the ability to capture bacteria from a food substance, apple juice was purchased from a local grocery store. S. aureus was grown and spiked into apple juice (as described in Examples 3 and 4). The CE was measured on pDA-MNPs by using plate counts and OD₆₀₀ values.

Capture of S. aureus was measured at many concentrations of MNCs, as was done in Example 6. When the MNC/CFU ratio was more than 4000, the CE was 0.93±0.016 (mean±95% confidence interval, n=21).

### Example 10—Growth in Presence of pDA-MNCs

The black-colored material extracted from a solution (i.e., the mixture of pDA-MNCs bound with bacteria) was cultured in the appropriate nutrient broth from at least one experiment for each bacterial species to see if the bacteria were still viable. No attempt was made to separate the MNPs from the bacteria; the black sample was dispersed, incubated at 37° C., and then sampled at specified times for plate counting. For every bacterial species investigated, the growth curve (measured over several hours by plate counts) was not different from that of non-captured bacteria, as assessed by the lag-phase time and the exponential growth rate constant. This suggests that the captured bacteria were still viable, and the pDA-MNPs, whether attached or adjacent in suspension, were not cytotoxic at the concentrations and conditions of these experiments.

Any and all of the components in the figures, embodiments, implementations, instances, cases, examples, methods, applications, iterations, and other parts of this disclosure can be combined, in whole or in part, and in any suitable manner. Additionally, any component can be removed, separated from other components, modified with or without modification of like components, or otherwise altered together or separately from anything else disclosed herein.

As used herein, the singular forms “a”, “an”, “the” and other singular references include plural referents, and plural references include the singular, unless the context clearly dictates otherwise. For example, reference to a pDA coating includes reference to one or more pDA coatings, and reference to MNPs includes reference to one or more MNPs. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object-regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. A method for extracting microorganisms from an extraction target, the method comprising:
forming a polymerized pDA coating on a substrate, the polymerized pDA coating comprising at least one of: (a) dopamine; and (b) a dopamine substitute;
exposing the polymerized pDA coating on the substrate to microorganisms such that at least a portion of the microorganisms binds to the polymerized pDA coating; and
collecting the polymerized pDA coating and the substrate together with the portion of the microorganisms that is bound to the polymerized pDA coating.

2. The method of claim 1, wherein the substrate comprises a magnetic nano particle, and wherein the method further comprises forming the magnetic nano particle.

3. The method of claim 1, wherein the exposing the polymerized pDA coating on the substrate to microorganisms comprises exposing the pDA coating on the substrate to a fluid.

4. The method of claim 3, wherein the fluid comprises a gas.

5. The method of claim 1, wherein the polymerized pDA coating comprises the dopamine substitute.

6. The method of claim 1, wherein the substrate comprises a microfluidic device.

7. The method of claim 1, wherein the substrate comprises a cluster of magnetic nano particles.

8. A composition for extracting microorganisms from an extraction target, the composition comprising:
a polymerized pDA coating comprising at least one of: (a) dopamine; and (b) a dopamine substitute.

9. The composition of claim 8, wherein the composition comprises the dopamine.

10. The composition of the claim 8, wherein the composition comprises the dopamine substitute.

11. The composition of claim 10, wherein the dopamine substitute comprises norepinephrine.

12. The composition of claim 10, wherein the composition further comprises the dopamine.

13. The composition of claim 8, further comprising an iron-oxide-based magnetic nano cluster, wherein the polymerized pDA coating is formed on a surface of the iron-oxide-based magnetic nano cluster.

14. The composition of claim 13, wherein a thickness of the polymerized pDA coating on the surface of the iron-oxide-based magnetic nano cluster is less than 8 nm.

15. An apparatus configured to extract microorganisms from an extraction target, the apparatus comprising:
a surface; and
a polymerized pDA coating formed on the surface, the polymerized pDA coating comprising at least one of: (a) dopamine; and (b) a dopamine substitute.

16. The apparatus of claim 15, further comprising a microscope slide comprising the surface.

17. The apparatus of claim 15, further comprising a microfluidic device having the surface formed on an interior thereof.

18. The apparatus of claim 15, further comprising a magnetic nano particle co-formed on the surface with the polymerized pDA coating.

19. The apparatus of claim 15, wherein the apparatus is configured to receive microorganisms from an environment and to capture a portion of the microorganisms in order to monitor a microbiology of the environment.

20. The apparatus of claim 15, wherein the apparatus is configured to selectively extract target microorganisms from the extraction target.
